(19) United States
(12) Patent Application Publication
(10) Pub. No.: US 2009/0317503 A1
(43) Pub. Date: Dec. 24, 2009

(54) Eyelid Preparation and Method of Use

(75) Inventors: Nat G. Adkins, JR., Richmond, TX (US); Cynthia Barratt, Richmond, TX (US)

Correspondence Address: D'AMBROSIO & ASSOCIATES, P.L.L.C., 10260 WESTHEIMER, SUITE 465, HOUSTON, TX, 77042, US

(73) Assignee: OCUSOFT, INC., Rosenburg, TX (US)

(21) Appl. No.: 12/487,552

(22) Filed: Jun. 18, 2009

Related U.S. Application Data
(60) Provisional application No. 61/132,583, filed on Jun. 20, 2008.

(51) Int. Cl. A61K 36/61; A61K 36/72; A61P 27/02

(52) U.S. Cl. 424/776; 424/725

(57) ABSTRACT

Embodiments of the invention comprise an eyelid preparation, a method for producing the eyelid preparation, a method for treating or preventing eyelid conditions and a method for applying an eyelid preparation on human eyelids or eyelashes. The eyelid preparation may be used to treat ocular conditions, such as blepharitis, dry eyes and dysfunctions of the meibomian glands, associated with an infestation of Demodex mites. The eyelid preparation consists essentially of therapeutically effective amounts of an essential oil and a dermatologically acceptable carrier.

### PRIORITY

This application claims priority to U.S. Provisional Patent Application Ser. Number 61/132,583 filed Jun. 20, 2008.

### BACKGROUND OF THE INVENTION

The eyelids are important to ocular health because they protect the eyes from airborne contaminants, such as pollen, dust particles or other foreign bodies. The eyelids contain several glands including the lacrimal glands and the specialized form of the sebaceous glands, the meibomian glands, which produce layers of tear film that are critical for healthy eyes.

The eyelid are subject to problems like blepharitis, dry eyes and inflammation of the meibomian glands. Another complication is the infestation of the arachnid, Demodex folliculorum (Demodex mites). The Demodex mite infestation is common in humans; anecdotal evidence suggests that the mites can be found in ten per cent of the eye lashes of healthy persons. The occurrence of the infestation may also be age related.

Demodex mites reside inside the sebaceous glands and hair follicles. They cause damage to the cell walls by sucking nutrients from the hair roots. They burrow into the skin, lay eggs, introduce bacteria, and infect the skin. Some of the symptoms of infestation include itching and inflammation of the eyelids. Additionally, there is evidence that the mites can also be one of the causes of the skin disease rosacea.

### SUMMARY OF THE INVENTION

Embodiments of the invention comprise an eyelid preparation, a method for producing the eyelid preparation, a method for treating or preventing eyelid conditions and a method for applying an eyelid preparation on human eyelids or eyelashes. The eyelid preparation is an ocular composition that may treat or prevent blepharitis, dry eyes, meibomianitis, and infestations of mite organisms.

In one embodiment of the invention, the eyelid preparation consists essentially of therapeutically effective amounts of an essential oil and a dermatologically acceptable carrier. In one embodiment of the invention, the essential oil comprises tea tree oil. In another embodiment of the invention, the essential oil comprises sea buckthorn oil. In one embodiment of the invention, the dermatologically acceptable carrier comprises petrolatum. In another embodiment of the invention, the dermatologically acceptable carrier comprises a gelling agent, the gelling agent comprising cellulose or a water soluble cellulose derivative. In yet another embodiment, the dermatologically acceptable carrier is selected from the group consisting of methyl cellulose, hydroxypropyl cellulose, methyl hydroxypropyl cellulose, hydroxypropyl methyl cellulose, cellulose acetate, ethyl cellulose, methyl hydroxyethyl cellulose, hydroxyethyl cellulose, and cellulose gum.

In one embodiment, the eyelid preparation consists essentially of therapeutically effective amounts of tea tree oil, petrolatum and mineral oil to treat or prevent ocular conditions selected from a group consisting of meibomian gland dysfunctions, blepharitis, dry eyes, and infestations of Demodex mites.

In another embodiment, the eyelid preparation consists essentially of therapeutically effective amounts of sea buckthorn oil, petrolatum and mineral oil to treat or prevent ocular conditions selected from a group consisting of meibomian gland dysfunctions, blepharitis, dry eyes, and infestations of Demodex mites.

In yet another embodiment, the eyelid preparation consists essentially of therapeutically effective amounts of tea tree oil and a cellulose derivative in an amount effective to treat or prevent ocular conditions selected from a group consisting of meibomian gland dysfunctions, blepharitis, dry eyes, and infestations of Demodex mites.

In another embodiment, the eyelid preparation consists essentially of therapeutically effective amounts of sea buckthorn oil and a cellulose derivative in an amount effective to treat or prevent ocular conditions selected from a group consisting of meibomian gland dysfunctions, blepharitis, dry eyes, and infestations of Demodex mites. The eyelid preparation may have a ratio by weight of the sea buckthorn oil to the cellulose derivative in a range comprising from 45:0.5 to 50:1.

The eyelid preparation may further comprise a preservative. In one embodiment, the preservative comprises sodium perborate or benzylkonium chloride.

In one or more embodiments, the eyelid preparation further comprises mineral oil.

The eyelid preparation may take the form of a gel, ointment, cream, lotion or paste. In one embodiment of the invention, the gel has a viscosity ranging from about 75,000 centipoise to about 3,000,000 centipoise.

In one embodiment, the eyelid preparation may consist of 0.5-1.5 parts of the dermatologically acceptable carrier to 45-55 parts of the essential oil.

In another embodiment, the eyelid preparation consists essentially of therapeutically effective amounts of tea tree oil, petrolatum and mineral oil to treat or prevent ocular conditions selected from a group consisting of meibomian gland dysfunctions, blepharitis, dry eyes, and infestations of Demodex mites.

In one or more embodiments, the eyelid preparation has a ratio by weight of the sea buckthorn oil to the cellulose derivative in a range comprising from 45:0.5 to 50:1.

In one embodiment of the invention, a fabric pad or other absorbent material is impregnated with the eyelid preparation, wherein the eyelid preparation comprises therapeutically effective amounts of an essential oil and a dermatologically acceptable carrier. The impregnated fabric pad may be used to treat blepharitis, dry eyes, meibomianitis, and infestations of mite organisms.

In another embodiment, a method for producing an eyelid preparation for treating or preventing ocular conditions selected from a group consisting of meibomian gland dysfunctions, blepharitis, dry eyes, and infestations of Demodex mites comprises mixing sea buckthorn oil and a cellulose derivative in therapeutically effective amounts. The eyelid preparation may be a gel having a viscosity ranging from about 75,000 cps to about 3,000,000 cps.

The method further comprises coating an applicator with the eyelid preparation. In one or more embodiments, the applicator may be sealed within an impervious pouch. The applicator may comprise a disposable apparatus. Exemplary applicators may comprise cotton swabs, fabric pads, brushes and cotton buds.

In another embodiment, a method for treating or preventing ocular conditions selected from a group consisting of meibomian gland dysfunctions, blepharitis, dry eyes, and infestations of Demodex mites comprises identifying a treatment site: coating an applicator with an eyelid preparation for application to and penetration into the treatment site, wherein the eyelid preparation comprises therapeutically effective amounts of an essential oil and a dermatologically acceptable carrier; dispensing the eyelid preparation to the treatment site with the applicator: and allowing the eyelid preparation to penetrate into the treatment site, wherein the eyelid preparation has a viscosity that allows rapid absorption into the treatment site while avoiding leakage of the eyelid preparation into the eye.

In yet another embodiment, a method for applying an eyelid preparation on human eyelids or eyelashes infected with Demodex mites comprises: coating an applicator with the eyelid preparation, the eyelid preparation comprising tea tree oil and a cellulose derivative in an amount effective to treat the eyelids or the eyelashes; manipulating the applicator for application of the eyelid preparation on the eyelids or the eyelashes, wherein the eyelid preparation is allowed to remain on the eyelids or the eyelashes for a pre-determined time period: and discarding the applicator after the application of the eyelid preparation is completed.

The eyelids or the eyelashes may be rinsed with saline after the pre-determined time period. In another embodiment, the eyelid preparation may be re-applied to the eyelids or the eyelashes for additional 5 minute-10 minute periods.

### DETAILED DESCRIPTION

The eyelid preparation of the invention is an ocular or ophthalmic composition. The eyelid preparation can be constituted to take the form of a gel, ointment, cream, lotion or paste. In some embodiments of this invention, the eyelid preparation consists essentially of therapeutically effective amounts of an essential oil and a dermatologically acceptable carrier for the essential oil. In other embodiments, the eyelid preparation further comprises trace amounts of mineral oil.

The essential oil is the active ingredient of the eyelid preparation. Essential oils are naturally occurring, hydrophobic liquids extracted from plants. In one embodiment, the essential oil or hydrophobic plant active comprises tea tree oil. Tea tree oil or melaleuca oil is an essential oil obtained by steam distilling the leaves of the Melaleuca allernifolia which is native to the northeast coast of New South Wales, Australia. Tea tree oil contains constituents called terpenoids, a naturally occurring organic chemical.

In another embodiment, the essential oil comprises sea buckthorn oil. Sea-buckthorn identifies a group of species in the genus Hippophae rhamnoides. Sea buckthorn oil can be extracted from either the seeds or the pulp of the fruit. The natural antioxidants and essential fatty acids in the sea buckthorn oil may help reverse damaging effects of sun radiation and minimize long term effects of sun exposure, like wrinkles, dryness, dark spots reduce skin inflammation, promote natural skin restorative processes.

A pharmacologically and dermatologically acceptable ingredient acts as carrier or vehicle for the essential oil. In one embodiment, the carrier is a hydrophobic carrier that is not easily washed away from the surface and can be applied to the skin and eyelids. In another embodiment, the carrier comprises an excipient or non-active ingredient containing at least 30% petrolatum. The term petrolatum is well understood in the food, drug and cosmetic industries to include alternative names such as “soft paraffin”, “petroleum jelly”, “refined petrolatum” or “purified petrolatum”. Petrolatum is a semi-solid mixture of hydrocarbons. Preferably, the petrolatum is refined to meet FDA requirements.

In another embodiment, the carrier comprises a gelling agent that provide a safe means for time release of the essential oils to the eyelids. The gelling agents comprise cellulosic substance selected from among, for example, cellulose, cellulose derivative and regenerated cellulose. In other embodiments, the carrier comprises a water soluble cellulose derivative. This cellulose derivate is selected from the group consisting of methyl cellulose, hydroxy propyl cellulose, methyl hydroxypropyl cellulose, hydroxy-propyl methyl cellulose, cellulose acetate, ethyl cellulose, methyl hydroxyethyl cellulose, hydroxy ethyl cellulose, and cellulose gum.

The eyelid preparation may also comprise a preservative, in other embodiments. A preservative is an agent that prolongs the useful life of a material. The preservative may additionally possess biocidal properties. A biocide is a substance that kills or inhibits the growth of microorganisms such as bacteria, molds, slimes, fungi, etc. In one embodiment, the preservative comprises sodium perborate or benzylkonium chloride.

Mites of the genus Demodex are commonly found infesting the area around hair follicles and sebaceous glands in mammals. Two species known to infest humans, include Demodex folliculorum. found on the hair follicles of humans and, Demodex brevis, infesting the sebaceous glands associated with the hair follicles. Together, these mites are commonly referred to as “eyelash mites”. The Demodex mites are quite small, adults being in the 0.3 mm to 0.4 mm range. Infestations of the mites on the eyelashes and eyelids are commonly observed. Infestation by Demodex mites is common especially in older adult humans.

An overpopulation of the mites may cause a condition known as demodicosis, which leads to inflammation of the skin, itching, and associated skin disorders. An inflammation of the eyelids known as blepharitis can also be caused by Demodex infestation. Often associated with or secondary to blepharitis is a bacterial infection of the surface of the skin at the edge of the lid, known as an internal hordeolum. Other such infections include external hordeolum, commonly referred to styes, which are infections of the tiny oil secreting meibomian glands along the edge of the eyelid, surrounding the eyelashes. A stye begins as a red, tender bump and usually fully develops within three days. Such conditions are accompanied by pain, redness and tenderness of the lid margins.

Embodiments of the invention include an eyelid preparation that may be effective as a parasiticide to treat or kill Demodex mites. In other embodiments, the eyelid preparation can treat or prevent ocular conditions such as dry eyes, blepharitis and meibomianitis or dysfunctions of the meibomian glands that may result from infestations of Demodex mites. The eyelid preparation is designed for application on human or animal skin and hair; particularly the hair and skin of the face, eyelids, and the area around the eyelids of humans.

The eyelid preparation consists of 0.5-1.5 parts of the dermatologically acceptable carrier to 45-55 parts of the essential oil. In one embodiment of the invention, the eyelid preparation has a ratio by weight of the tea tree oil or sea buckthorn oil to the cellulose derivative in a range comprising from 45:0.5 to 50:1. The viscosity of the eyelid preparation may be adjusted so that the composition is rapidly absorbed by the eyelids and so that the essential oil does not drip or run into the eyes. In other embodiments of the invention, the gel viscosity may have a range of about 75,000 centipoise to about 3,000,000 centipoise to aid the absorption of the eyelid preparation.

In another embodiment of the invention, a method for producing an eyelid preparation comprises mixing an essential oil and a dermatologically acceptable carrier in therapeutically effective amounts. In other embodiments, trace amounts of mineral oil may be added to the mixture. In yet other embodiments, a suitable preservative may be added to increase the shelf-life of the composition.

In another embodiment, a method for treating or preventing ocular conditions is also disclosed. The method for treating or preventing ocular conditions comprises identifying a treatment site. The treatment site may be the eyes or the skin or hair follicles surrounding the eyelids. An applicator may be coated with the eyelid preparation (described earlier) consisting of an essential oil and a dermatologically acceptable carrier in therapeutically effective amounts for application to and penetration into the treatment site. Single-use or multi-use applicators may be impregnated with or coated with the eyelid preparation. Various types of applicators may therefore be used, including, but not limited to, brushes, cotton swabs, and polymer meshes. Once treatment is completed, the applicator may be discarded and the treated area rinsed to remove any excess chemicals.

In one embodiment, the applicator comprises a fabric pad. A person skilled in the art will recognize that numerous fabric and fabric blends may be chosen for the applicator. The fabric may be natural, synthetic, or a blended weave. The ideal fabric is gentle enough to be used on the eyelids and the sensitive skin surrounding the eyes, but is also rough enough or textured enough to provide a significantly tactile surface to effectively treat the eyelids. Furthermore, the fabric pad must have sufficient loft and interstitial spaces to hold the eyelid preparation in the fabric weave long enough to effectively apply the eyelid preparation to the treatment site.

The fabric pads may be of a disposable or reusable nature. In one embodiment, fabric pads are pre-coated with the eyelid preparation and are placed in a sealed pouch or sealable container to protect the contents of the pouch from the environment. The sealable container may further comprise a box, or a package. The package may be made of any suitable material including plastic or a metal foil material. The pre-coated fabric pads may be individually packaged for use.

The applicator may be disposable, as discussed herein. Alternately, the thumb or other fingers may be used to apply the eyelid preparation of the invention. The eyelid preparation is dispensed to the treatment site with the applicator. The eyelid preparation is designed to stay on the treatment site long enough for the active ingredient, the essential oil, to penetrate and to be properly absorbed, but without lingering too long on the skin prior to absorption, thus causing a feeling or “greasiness”, uneasiness, or discomfort to patient. In one embodiment, the eyelid preparation has a viscosity that allows rapid absorption into the treatment site while avoiding leakage of the eyelid preparation into the eye.

In another embodiment, a method for applying an eyelid preparation on human eyelids or eyelashes infected with Demodex mites comprises coating an applicator with the eyelid preparation. The eyelid preparation comprises an essential oil, such as tea tree oil or sea buckthorn oil, and a cellulose derivative in an amount effective to treat or kill the Demodex mites. The applicator is manipulated for application of the eyelid preparation on the eyelids or the eyelashes. The eyelid preparation is allowed to remain on the eyelids or the eyelashes for a pre-determined time period. The applicator is discarded after the application of the eyelid preparation is completed.

The eyelid preparation is readily removable and washable without the use of harsh or damaging cleansers. In one embodiment, the eyelids or the eyelashes are rinsed with saline after the pre-determined time period. The pre-determined time period can range from 5 minutes to 10 minutes. In other aspects, the eyelid preparation is reapplied after a rest period. The rest period can range from 5 minutes to 20 minutes. Following the rest period, the eyelid preparation is reapplied for another 5-10 minute period. This treatment is repeated weekly until the Demodex infestation is completely destroyed.

While the invention has been described with reference to several embodiments, it will be understood by those skilled in the art that various changes in composition, form and detail may be made therein without departing from the spirit and scope of the invention. Thus, it is intended that the present invention cover the modifications and variations of this invention provided that they come within the scope of any claims and their equivalents.

1. An eyelid preparation for treating ocular conditions consisting essentially of:
therapeutically effective amounts of an essential oil and a dermatologically acceptable carrier.

2. The eyelid preparation of claim 1, wherein the essential oil comprises tea tree oil.

3. The eyelid preparation of claim 1, wherein the essential oil comprises sea buckthorn oil.

4. The eyelid preparation of claim 1, wherein the dermatologically acceptable carrier comprises petrolatum.

5. The eyelid preparation of claim 1, wherein the dermatologically acceptable carrier comprises a gelling agent, the gelling agent comprising cellulose or a water soluble cellulose derivative.

6. The eyelid preparation of claim 5, wherein the dermatologically acceptable carrier is selected from the group consisting of methyl cellulose, hydroxypropyl cellulose, methyl hydroxypropyl cellulose, hydroxypropyl methyl cellulose, cellulose acetate, ethyl cellulose, methyl hydroxyethyl cellulose, hydroxyethyl cellulose, and cellulose gum.

7. The eyelid preparation of claim 1 further comprising a preservative.

8. The eyelid preparation of claim 7, wherein the preservative comprises sodium perborate or benzylkonium chloride.

9. The eyelid preparation of claim 1 further comprising mineral oil.

10. The eyelid preparation of claim 1, wherein the eyelid preparation is a gel, ointment, cream, lotion or paste.

11. The eyelid preparation of claim 1, wherein the eyelid preparation is an ocular composition that treats or prevents blepharitis, dry eyes, meibomianitis, and infestations of mite organisms.

12. The eyelid preparation of claim 11, wherein the mite organisms comprise Demodex folliculorum.

13. The eyelid preparation of claim 1, wherein the eyelid preparation consists of 0.5-1.5 parts of the dermatologically acceptable carrier to 45-55 parts of the essential oil.

14. A method for producing an eyelid preparation, the method comprising combining an essential oil in a dermatologically acceptable carrier, wherein the eyelid preparation consists of 0.5-1.5 parts of the dermatologically acceptable carrier to 45-55 parts of the essential oil.

15. The method of claim 14, wherein the essential oil comprises tea tree oil.

16. The method of claim 14, wherein the essential oil comprises sea buckthorn oil.

17. The method of claim 14, wherein the dermatologically acceptable carrier comprises petrolatum.

18. The method of claim 14, wherein the dermatologically acceptable carrier comprises cellulose or a water soluble cellulose derivative.

19. The method of claim 14, further comprising impregnating a fabric pad with the eyelid preparation produced in claim 1.

20. A method for producing an eyelid preparation for treating or preventing ocular conditions selected from a group consisting of meibomian gland dysfunctions, blepharitis, dry eyes, and infestations of Demodex mites, the method comprising mixing sea buckthorn oil and a cellulose derivative in therapeutically effective amounts, wherein the eyelid preparation is a gel having a viscosity ranging from about 75,000 cps to about 3,000,000 cps.

21. The method of claim 20, further comprising coating an applicator with the eyelid preparation produced in claim 1.

22. The method of claim 21, further comprising sealably enclosing the applicator within an impervious pouch.

23. The method of claim 21, wherein the applicator comprises a disposable apparatus.

24. A method for treating or preventing ocular conditions selected from a group consisting of meibomian gland dysfunctions, blepharitis, dry eyes, and infestations of Demodex mites comprising:
identifying a treatment site:
coating an applicator with an eyelid preparation for application to and penetration into the treatment site, the eyelid preparation comprising therapeutically effective amounts of an essential oil and a dermatologically acceptable carrier;
dispensing the eyelid preparation to the treatment site with the applicator;
and
allowing the eyelid preparation to penetrate into the treatment site, wherein the eyelid preparation has a viscosity that allows rapid absorption into the treatment site while avoiding leakage of the eyelid preparation into the eye.

25. The method of claim 24, wherein the essential oil comprises tea tree oil.

26. The method of claim 24, wherein the dermatologically acceptable carrier comprises a cellulose derivative.

27. The method of claim 24, wherein an eyelid preparation ratio of the essential oil to the cellulose derivative is 50:1.

28. A method for applying an eyelid preparation on human eyelids or eyelashes infected with Demodex mites comprising:
coating an applicator with the eyelid preparation, the eyelid preparation comprising tea tree oil and a cellulose derivative in an amount effective to treat the eyelids or the eyelashes;
manipulating the applicator for application of the eyelid preparation on the eyelids or the eyelashes, wherein the eyelid preparation is allowed to remain on the eyelids or the eyelashes for a pre-determined time period; and
discarding the applicator after the application of the eyelid preparation is completed.

29. The method of claim 28, further comprising rinsing the eyelids or the eyelashes with saline after the pre-determined time period.

30. The method of claim 29, further comprising reapplying the eyelid preparation to the eyelids or the eyelashes for additional 5 minute-10 minute periods.
